(19) United States
(12) Patent Application Publication
(10) Pub. No.: US 2009/0069669 A1
(43) Pub. Date: Mar. 12, 2009

(54) Efficient Features for Detection of Motion Artifacts in Breast MRI

(75) Inventors: Gerardo Hermosillo Valadez, West Chester, PA (US); Yoshihisa Shinagawa, Downingtown, PA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc., Malvern, PA (US)

(21) Appl. No.: 12/204,907

(22) Filed: Sep. 5, 2008

Related U.S. Application Data
(60) Provisional application No. 60/971,344, filed on Sep. 11, 2007.

(51) Int. Cl. A61B 5/055

(52) U.S. Cl. 600/420

(57) ABSTRACT

A method for identifying motion artifacts in a dynamic contrast enhanced MRI includes receiving a dynamic contrast enhanced MRI including a patient's breast on which motion correction has been performed. One or more regions of suspicion are automatically identified within the breast based in the dynamic contrast enhanced MRI. The regions of suspicion are examined. A measure of negative enhancement is calculated within a local neighborhood about each identified region of suspicion. Each identified region of suspicion for which the calculated measure of negative enhancement is greater than a predetermined threshold is removed.

### CROSS-REFERENCE TO RELATED APPLICATION

The present application is based on provisional application Ser. No. 60/971,344 filed Sep. 11, 2007, the entire contents of which are herein incorporated by reference.

### BACKGROUND OF THE INVENTION

1. Technical Field

The present disclosure relates to breast MRI and, more specifically, to efficient features for detection of motion artifacts in breast MRI.

2. Discussion of Related Art

Computer aided diagnosis (CAD) is the process of using computer vision systems to analyze medical image data and make a determination as to what regions of the image data are potentially problematic. Some CAD techniques then present these regions of suspicion to a medical professional such as a radiologist for manual review, while other CAD techniques make a preliminary determination as to the nature of the region of suspicion. For example, some CAD techniques may characterize each region of suspicion as a lesion or a non-lesion. The final results of the CAD system may then be used by the medical professional to aid in rendering a final diagnosis.

Because CAD techniques may identify lesions that may have been overlooked by a medical professional working without the aid of a CAD system, and because CAD systems can quickly direct the focus of a medical professional to the regions most likely to be of diagnostic interest, CAD systems may be highly effective in increasing the accuracy of a diagnosis and decreasing the time needed to render diagnosis. Accordingly, scarce medical resources may be used to benefit a greater number of patients with high efficiency and accuracy.

CAD techniques have been applied to the field of mammography, where low-dose x-rays are used to image a patient's breast to diagnose suspicious breast lesions. However, because mammography relies on x-ray imaging, mammography may expose a patient to potentially harmful ionizing radiation. As many patients are instructed to undergo mammography on a regular basis, the administered ionizing radiation may, over time, pose a risk to the patient. Moreover, it may be difficult to use x-rays to differentiate between different forms of masses that may be present in the patient's breast. For example, it may be difficult to distinguish between calcifications and malignant lesions.

Magnetic resonance imaging (MRI) is a medical imaging technique that uses a powerful magnetic field to image the internal structure and certain functionality of the human body. MRI is particularly suited for imaging soft tissue structures and is thus highly useful in the field of oncology for the detection of lesions.

In dynamic contrast enhanced MRI (DCE-MRI), many additional details pertaining to bodily soft tissue may be observed. These details may be used to further aid in diagnosis and treatment of detected lesions.

DCE-MRI may be performed by acquiring a sequence of MR images that span a time before magnetic contrast agents are introduced into the patient's body and a time after the magnetic contrast agents are introduced. For example, a first MR image may be acquired prior to the introduction of the magnetic contrast agents, and subsequent MR images may be taken at a rate of one image per minute for a desired length of time. By imaging the body in this way, a set of images may be acquired that illustrate how the magnetic contrast agent is absorbed and washed out from various portions of the patient's body. This absorption and washout information may be used to characterize various internal structures within the body and may provide additional diagnostic information.

However, even though the patient may be provided with instructions to remain completely still while the set of images is acquired, some amount of movement is inevitable, and image processing techniques may be used to compensate for patient motion. These techniques may employ rigid and non-rigid transformations to align the various images of the DCE-MRI sequence to compensate for patient movement so that absorption and washout may be accurately observed.

These techniques for compensating for patient motion may introduce artifacts into the compensated images. Then, when the DCE-MRI sequence is analyzed to identify suspicious lesions, motion artifacts may be misidentified as suspicious lesions.

### SUMMARY

A method for identifying motion artifacts in a dynamic contrast enhanced MRI includes receiving a dynamic contrast enhanced MRI including a patient's breast on which motion correction has been performed. One or more regions of suspicion are automatically identified within the breast based in the dynamic contrast enhanced MRI. A measure of negative enhancement is calculated within a local neighborhood about each identified region of suspicion. Each identified region of suspicion for which the calculated measure of negative enhancement is greater than a predetermined threshold is removed.

The dynamic contrast enhanced MRI may include a pre-contrast MR image and a sequence of post-contrast MR images acquired at a regular interval of time after administration of a magnetic contrast agent. The automatic identification of the regions of suspicion within the breast may include identifying the regions of suspicion based on an absorption and washout profile observed from the dynamic contrast enhanced MRI.

The dynamic contrast enhanced MRI may be corrected for magnetic field inhomogeneity prior to identifying the regions of suspicion. Segmentation of the breast may be performed on the dynamic contrast enhanced MRI prior to identifying the regions of suspicion.

A method for automatically detecting breast lesions includes acquiring a pre-contrast magnetic resonance (MR) image including a patient's breast. A magnetic contrast agent is administered. A sequence of post-contrast MR images including the patient's breast is acquired. Motion correction is performed on the sequence of post-contrast MR images. One or more regions of suspicion are automatically identified within the breast. One or more false positives are removed from the one or more regions of suspicion to generate a set of remaining regions of suspicion by determining which of the one or more regions of suspicion are the product of motion artifacts caused by the performance of motion correction. The set of remaining regions of suspicion is displayed.

The pre-contrast MR image and the sequence of post-contrast MR images may be part of a dynamic contrast enhanced MRI. The sequence of post-contrast MR images may be acquired at a regular interval of time after the administration of the contrast agent. The regular interval of time may be one image per minute.

The pre-contrast MR image may include T1 and T2 relaxation modalities. The sequence of post-contrast MR images may include a T1 relaxation modality.

The automatic identification of the regions of suspicion within the breast includes identifying the regions of suspicion based on an absorption and washout profile observed from the sequence of post-contrast MR images.

The pre-contrast MR image and the sequence of post-contrast MR images may be corrected for magnetic field inhomogeneity prior to identifying the regions of suspicion. Segmentation of the breast may be performed on the pre-contrast MR image and the sequence of post-contrast MR images prior to identifying the regions of suspicion.

One or more of the identified regions of interest may be automatically characterized according to a BIRADS classification based on an absorption and washout profile for the respective identified region of suspicion observed from the sequence of post-contrast MR images.

The step of removing one or more false positives from the one or more regions of suspicion may include examining each identified region of suspicion, calculating a measure of negative enhancement within a local neighborhood about each identified region of suspicion, and removing each identified region of suspicion for which the calculated measure of negative enhancement is greater than a predetermined threshold.

A computer system includes a processor and a program storage device readable by the computer system, embodying a program of instructions executable by the processor to perform method steps for automatically detecting breast lesions, the method includes receiving a dynamic contrast enhanced MRI including a patient's breast on which motion correction has been performed. One or more regions of suspicion are automatically identified within the breast. One or more false positives are removed from the one or more regions of suspicion to generate a set of remaining regions of suspicion by determining which of the one or more regions of suspicion are the product of motion artifacts caused by the performance of motion correction. The set of remaining regions of suspicion are displayed.

The dynamic contrast enhanced MRI may includes a pre-contrast MR image and a sequence of post-contrast MR images acquired at a regular interval of time after administration of a magnetic contrast agent. The automatic identification of the regions of suspicion within the breast may include identifying the regions of suspicion based on an absorption and washout profile observed from the dynamic contrast enhanced MRI.

The step of removing one or more false positives from the one or more regions of suspicion may include examining each identified region of suspicion, calculating a measure of negative enhancement within a local neighborhood about each identified region of suspicion and removing each identified region of suspicion for which the calculated measure of negative enhancement is greater than a predetermined threshold.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings, wherein:

FIG. 1 is a flow chart illustrating a method for imaging a patient's breast using DCE-MRI and rendering a computer-aided diagnosis according to an exemplary embodiment of the present invention;

FIG. 2 is a set of graphs illustrating a correspondence between absorption and washout profiles for various BIRADS classifications according to an exemplary embodiment of the present invention;

FIG. 3 illustrates an example of the ridge and valley effect caused by motion artifacts;

FIG. 4 is a flow chart illustrating a method for identifying and removing false positives associated with motion artifacts in breast MR images according to exemplary embodiments of the present invention; and

FIG. 5 shows an example of a computer system capable of implementing the method and apparatus according to embodiments of the present disclosure.

### DETAILED DESCRIPTION OF THE DRAWINGS

In describing exemplary embodiments of the present disclosure illustrated in the drawings, specific terminology is employed for sake of clarity. However, the present disclosure is not intended to be limited to the specific terminology so selected, and it is to be understood that each specific element includes all technical equivalents which operate in a similar manner

Exemplary embodiments of the present invention seek to image a patient's breast using DCE-MRI techniques and then perform CM to identify regions of suspicion that are more likely to be malignant breast lesions. By utilizing DCE-MRI rather than mammography, additional data pertaining to contrast absorption and washout may be used to accurately distinguish between benign and malignant breast masses.

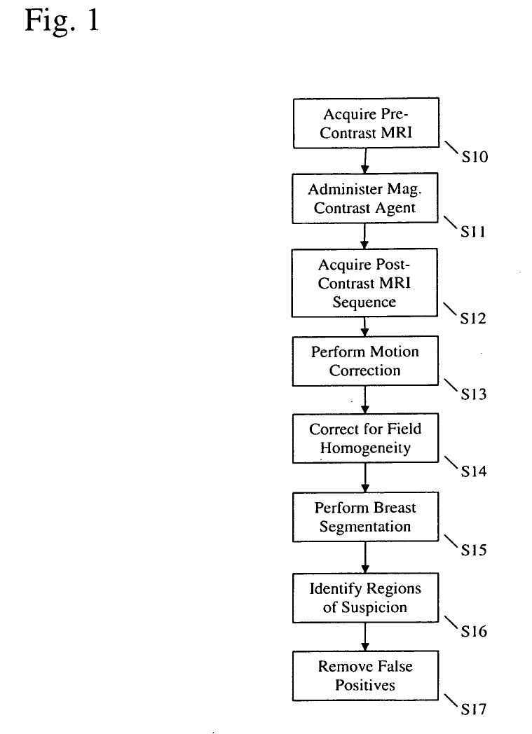

FIG. 1 is a flow chart illustrating a method for imaging a patient's breast using DCE-MRI and rendering a computer-aided diagnosis according to an exemplary embodiment of the present invention. First, a pre-contrast MRI is acquired (Step S10). The pre-contrast MRI may include an MR image taken of the patient before the magnetic contrast agent has been administered. The pre-contrast MRI may include one or more modalities. For example, both T1 and T2 relaxation modalities may be acquired.

Next, with the patient remaining as still as possible, the magnetic contrast agent may be administered (Step S11). The magnetic contrast agent may be a paramagnetic agent, for example, a gadolinium compound. The agent may be administered orally, intravenously, or by another means. The magnetic contrast agent may be selected for its ability to appear extremely bright when imaged in the T1 modality. By injecting the magnetic contrast agent into the patient's blood, vascular tissue may be highly visible in the MRI. Because malignant tumors tend to be highly vascularized, the use of the magnetic contrast agent may be highly effective for identifying regions suspected of being lesions.

Moreover, additional information may be gleamed by analyzing the way in which a region absorbs and washes out the magnetic contrast agent. For this reason, a sequence of post-contrast MR images may be acquired (Step S12). The sequence may be acquired at regular intervals in time, for example, a new image may be acquired every minute.

As discussed above, the patient may be instructed to remain as still as possible throughout the entire image acquisition sequence. Despite these instructions, the patient will most likely move somewhat from image to image. Accordingly, before regions of suspicion are identified (Step S16), motion correction may be performed on the images (Step S13).

At each acquisition, the image may be taken in the T1 modality that is well suited for monitoring the absorption and washout of the magnetic contrast agent.

Because MR images are acquired using a powerful magnetic field, subtle inhomogeneity in the magnetic field may have an impact on the image quality and may lead to the introduction of artifacts. Additionally, the level of enhancement in the post-contrast image sequence may be affected. Also, segmentation of the breast may be impeded by the inhomogeneity, as in segmentation, it is often assumed that a particular organ appears homogeneously. Accordingly, the effects of the inhomogeneous magnetic field may be corrected for within all of the acquired MR images (Step S14).

The order in which motion correction (Step S13) and inhomogeneity correction (Step S14) are performed on the MR images is not critical. All that is required is that these steps be performed after image acquisitions for each given image, and prior to segmentation (Step S15). These corrective steps may be performed for each image after each image is acquired or for all images after all images have been acquired.

After the corrective steps (Steps S13 and S14) have been performed, breast segmentation may be performed (Step S15). Segmentation is the process of determining the contour delineating a region of interest from the remainder of the image. In making this determination, edge information and shape information may be considered.

Edge information pertains to the image intensity changes between the interior and exterior of the contour. Shape information pertains to the probable shape of the contour given the nature of the region of interest being segmented. Some techniques for segmentation such as the classical watershed transformation rely entirely on edge information. Examples of this technique may be found in L. Vincent and P. Soille, “Watersheds in digital spaces: An efficient algorithm based immersion simulations” IEEE Trans, PAMI, 13(6):583-589, 1991, which is incorporated by reference. Other techniques for segmentation rely entirely on shape information. For example, in M. Kass, A. Witkin, and D. Terzopoulous, “Snakes—Active contour models” Int J. Comp Vis, 1(4): 321-331, 1987, which is incorporated by reference, a calculated internal energy of the curvature is regarded as a shape prior although its weight is hard-coded and not learned through training. In A. Tsai, A. Yezzi, W. Wells, C. Tempany, D. Tucker, A. Fan, and W. E. Grimson, “A shape-based approach to the segmentation of medical imagery using level sets” IEEE Trans. Medical Imaging, 22(2): 137-154, 2003, which is incorporated by reference, the shape prior of signed distance representations called eigenshapes is extracted by Principal Component Analysis (PCA). When the boundary of an object is unclear and or noisy, the shape prior is used to obtain plausible delineation.

When searching for lesions in the breast using DCE-MRI, internal structures such as the pectoral muscles that are highly vascularized may light up with the application of the magnetic contrast agent. Thus, the pectoral muscles, and other such structures may make location of breast lesions more difficult. Accordingly, by performing accurate segmentation, vascularized structures that are not associated with the breast tissue may be removed from consideration thereby facilitating fast and accurate detection of breast lesions.

After segmentation has been performed (Step S15), the breast tissue may be isolated and regions of suspicion may be automatically identified within the breast tissue region (Step S16). A region of suspicion is a structure that has been determined to exhibit one or more properties that make it more likely to be a breast lesion than the regions of the breast tissue that are not determined to be regions of suspicion. Detection of the region of suspicion may be performed by systematically analyzing a neighborhood of voxels around each voxel of the image data to determine whether or not the voxel should be considered part of a region of suspicion. This determination may be made based on the acquired pre-contrast MR image as well as the post-contrast MR image. Such factors as size and shape may be considered.

Moreover, the absorption and washout profile of a given region may be used to determine whether the region is suspicious. This is because malignant tumors tend to show a rapid absorption followed by a rapid washout. This and other absorption and washout profiles can provide significant diagnostic information.

Breast imaging reporting and data systems (BIRADS) is a system that has been designed to classify regions of suspicion that have been manually detected using conventional breast lesion detection techniques such as mammography and breast ultrasound. Under this approach, there are six categories of suspicious regions. Category 0 indicates an incomplete assessment. If there is insufficient data to accurately characterize a region, the region may be assigned to category 0. A classification as category 0 generally implies that further imaging is necessary. Category 1 indicates normal healthy breast tissue. Category 2 indicates benign or negative. In this category, any detected masses such as cysts or fibroadenomas are determined to be benign. Category 3 indicates that a region is probably benign, but additional monitoring is recommended. Category 4 indicates a possible malignancy. In this category, there are suspicious lesions, masses or calcifications and a biopsy is recommended. Category 5 indicates that there are masses with an appearance of cancer and biopsy is necessary to complete the diagnosis. Category 6 is a malignancy that has been confirmed through biopsy.

Exemplary embodiments of the present invention may be able to characterize a given region according to the above BIRADS classifications based on the DCE-MRI data. To perform this categorization, the absorption and washout profile, as gathered from the post-contrast MRI sequence, for each given region may be compared against a predetermined understanding of absorption and washout profiles.

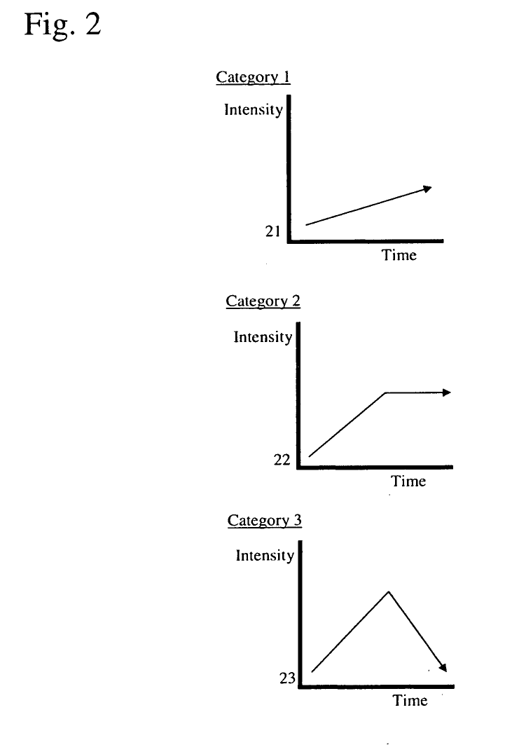

FIG. 2 is a set of graphs illustrating a correspondence between absorption and washout profiles for various BIRADS classifications according to an exemplary embodiment of the present invention. In the first graph 21, the T1 intensity is shown to increase over time with little to no decrease during the observed period. This behavior may correspond to a gradual or moderate absorption with a slow washout. This may be characteristic of normal breast tissue and accordingly, regions exhibiting this profile may be classified as category 1.

In the next graph 22, the T1 intensity is shown to increase moderately and then substantially plateau. This behavior may correspond to a moderate to rapid absorption followed by a slow washout. This may characterize normal breast tissue or a benign mass and accordingly, regions exhibiting this profile may be classified as category 2.

In the next graph 23, the T1 intensity is shown to increase rapidly and then decrease rapidly. This behavior may correspond to a rapid absorption followed by a rapid washout. While this behavior may not establish a malignancy, it may raise enough suspicion to warrant a biopsy, accordingly, regions exhibiting this profile may be classified as category 3.

Other absorption and washout profiles may be similarly established for other BIRAD categories. In this way, DCE-MRI data may be used to characterize a given region according to the BIRADS classifications. This and potentially other criteria, such as size and shape, may thus be used to identify regions of suspicion (Step S16).

After regions of suspicion have been identified, false positives may be removed (Step S17). As described above, artifacts such as motion compensation artifacts, artifacts cause by magnetic field inhomogeneity, and other artifacts, may lead to the inclusion of one or more false positives. Exemplary embodiments of the present invention and/or conventional approaches may be used to reduce the number of regions of suspicion that have been identified due to an artifact, and thus false positives may be removed. Removal of false positives may be performed by systematically reviewing each region of suspicion multiple times, each time for the purposes of removing a particular type of false positive. Each particular type of false positive may be removed using an approach specifically tailored to the characteristics of that form of false positive. Examples of such approaches are discussed in detail below.

After false positives have been removed (Step S17), the remaining regions of suspicion may be presented to the medical practitioner for further review and consideration. For example, the remaining regions of interest may be highlighted within a representation of the medical image data. Quantitative data such as size and shape measurements and/or BIRADS classifications may be presented to the medical practitioner along with the highlighted image data. The presented data may then be used to determine a further course of testing or treatment. For example, the medical practitioner may use the presented data to order a biopsy or refer the patient to an oncologist for treatment.

As discussed above, motion artifacts may be generated during the step of performing motion correction (Step S13). This may be the case regardless of what methods and algorithms are chosen to implement motion correction. In detecting lesions as areas of enhancement in dynamic contrast enhanced magnetic resonance imaging (DCE-MRI), motion artifacts may represent a significant portion of false positives. The resulting motion artifacts may be classified by the CAD system as regions of suspicion during identification (Step S16). Accordingly, the motion artifacts that are inadvertently characterized as regions of suspicion may burden the reviewing medical practitioner, reduce diagnostic efficiency and accuracy, and may potentially lead to unwarranted biopsy.

Exemplary embodiments of the present invention attempt to remove breast lesion false positives that are the result of motion artifacts by exploiting discovered characteristics that motion artifacts tend to share. The removal of false positives resulting from motion artifacts may be performed as part of the removal step discussed above (Step S17).

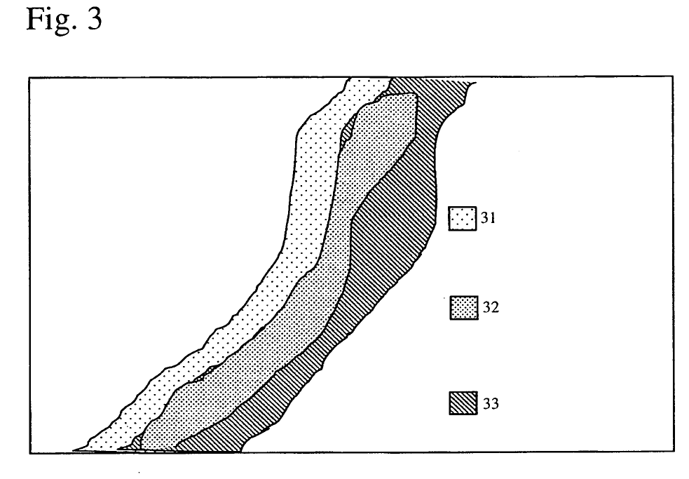

It has been discovered that motion artifacts tend to produce a ridge and valley effect. According to this effect, an enhancement is produced through misalignment of some anatomical structures or organs due to motion. This enhancement may be coupled with a dropout at the opposite side of the structure. FIG. 3 illustrates an example of the ridge and valley effect caused by motion artifacts. In this figure a blood vessel is shown in three different levels of enhancement 31, 32, and 33. Here, the vessel is shown as a bright structure over a dark background that is slightly misaligned as a result of motion between a first and second image capture. Because the structure is brighter than the background, the left portion 31 may appear as an area of spurious positive enhancement due to misalignment. The right portion 33 may appear as an area of spurious negative enhancement due to misalignment. This negative enhancement area represents the dropout discussed above. The middle portion 32 represents the area of intersection of the vessel seen in both the first and second images.

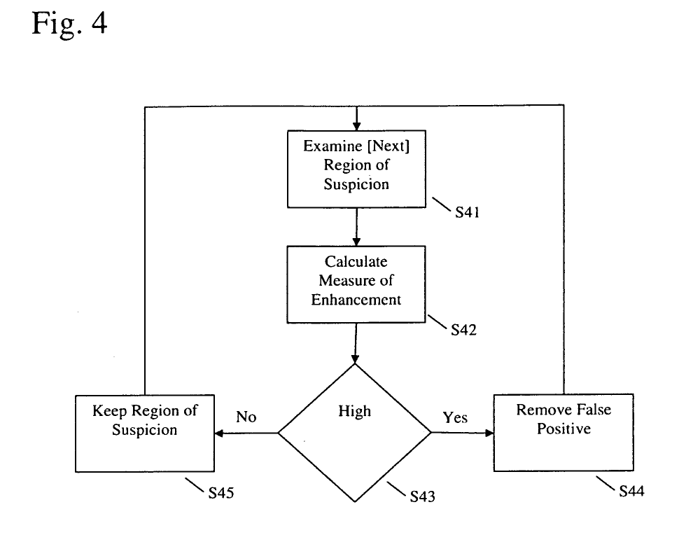

FIG. 4 is a flow chart illustrating a method for identifying and removing false positives associated with motion artifacts in breast MR images according to exemplary embodiments of the present invention. Each of the identified regions of suspicion may be examined for example, one-by-one. Accordingly, a first region of suspicion may be examined (Step S41). The region of suspicion may represent a region of positive enhancement. A local neighborhood around the region of suspicion may be examined to calculate a measure of negative enhancement or drop out in the local neighborhood around the region of positive enhancement of the region of suspicion (Step S42). For regions of suspicion that are positively enhanced due to motion artifact, the calculated measure of negative enhancement would be expected to be high. Accordingly, the calculated measure of negative enhancement is compared to a predetermined threshold (Step S43). If the calculated measure of negative enhancement is higher than the threshold (Yes, Step S43) then the corresponding region of suspicion may be regarded as a false positive and removed from the set of regions of suspicion (Step S44). If, however, the calculated measure of negative enhancement is lower than the threshold (No, Step S43) then the corresponding region of suspicion is preserved (Step S45).

The measure M of the negative enhancement around a given location y in an image I may be calculated as follows:

$$M = {\sum }_{x ∈ V ( y )} N ( I ( x ) )$$

where V(y) ⊂ [symbol] ={x:|x−y|<d} is the neighborhood of the region of suspicion for some norm |·|, d is a distance threshold and N: [symbol] selects the negative enhancement:

$$N ( x ) = \{ \begin{matrix}1 & if x < 0 \\ 0 & otherwise\end{matrix} \qquad ( 2 )$$

The distance threshold d, as well as the value Mₘₐₓ above which M is considered to be caused by a false positive may be determined using standard machine learning algorithms from a set of positive and negative examples. For example, if P⁺: [symbol] represents an estimate of the distribution of M among positive examples, and P⁻: [symbol] represents an estimate of the distribution of M among negative examples, the threshold Mₘₐₓ can be determined as the value above which P⁻(M)>P⁺(M).

This procedure may be repeated for each region of suspicion until all of the regions of suspicion have been examined. Because there may be multiple causes for false positives, each region of suspicion may be examined for each particular cause, and thus the procedure discussed above for locating and removing false positives that are the result of motion artifacts in breast MR may be combined with other procedures for removing other forms of false positives.

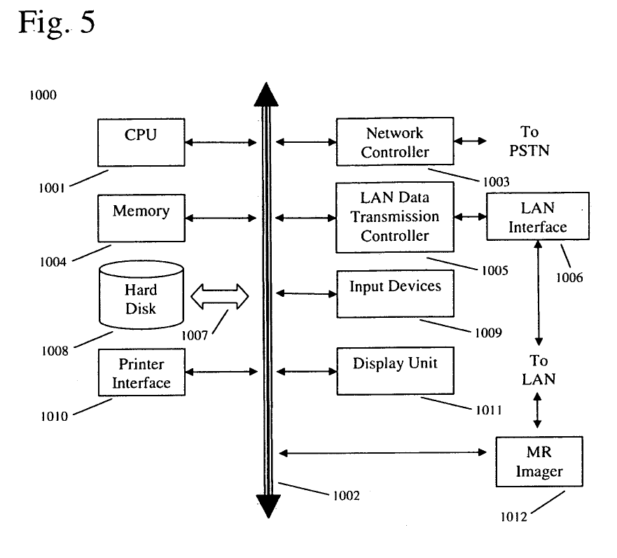

FIG. 5 shows an example of a computer system which may implement a method and system of the present disclosure. The system and method of the present disclosure may be implemented in the form of a software application running on a computer system, for example, a mainframe, personal computer (PC), handheld computer, servers etc. The software application may be stored on a recording media locally accessible by the computer system and accessible via a hard wired or wireless connection to a network, for example, a local area network, or the Internet.

The computer system referred to generally as system 1000 may include, for example, a central processing unit (CPU) 1001, random access memory (RAM) 1004, a printer interface 1010, a display unit 1011, a local area network (LAN) data transmission controller 1005, a LAN interface 1006, a network controller 1003, an internal bus 1002, and one or more input devices 1009, for example, a keyboard, mouse etc. As shown, the system 1000 may be connected to a data storage device, for example, a hard disk, 1008 via a link 1007. A MR imager 1012 may be connected to the internal bus 1002 via an external bus (not shown) or over a local area network.

Exemplary embodiments described herein are illustrative, and many variations can be introduced without departing from the spirit of the disclosure or from the scope of the appended claims. For example, elements and/or features of different exemplary embodiments may be combined with each other and/or substituted for each other within the scope of this disclosure and appended claims.

What is claimed is:

1. A method for identifying motion artifacts in a dynamic contrast enhanced MRI, comprising:
receiving a dynamic contrast enhanced MRI including a patient's breast on which motion correction has been performed;
automatically identifying one or more regions of suspicion within the breast based in the dynamic contrast enhanced MRI;
calculating a measure of negative enhancement within a local neighborhood about each identified region of suspicion; and
removing each identified region of suspicion for which the calculated measure of negative enhancement is greater than a predetermined threshold.

2. The method of claim 1, wherein the dynamic contrast enhanced MRI includes a pre-contrast MR image and a sequence of post-contrast MR images acquired at a regular interval of time after administration of a magnetic contrast agent.

3. The method of claim 1, wherein the automatic identification of the regions of suspicion within the breast include identifying the regions of suspicion based on an absorption and washout profile observed from the dynamic contrast enhanced MRI.

4. The method of claim 1, wherein the dynamic contrast enhanced MRI is corrected for magnetic field inhomogeneity prior to identifying the regions of suspicion.

5. The method of claim 1, wherein segmentation of the breast is performed on the dynamic contrast enhanced MRI prior to identifying the regions of suspicion.

6. A method for automatically detecting breast lesions, comprising:
acquiring a pre-contrast magnetic resonance (MR) image including a patient's breast;
administering a magnetic contrast agent;
acquiring a sequence of post-contrast MR images including the patient's breast;
performing motion correction on the sequence of post-contrast MR images;
automatically identifying one or more regions of suspicion within the breast;
removing one or more false positives from the one or more regions of suspicion to generate a set of remaining regions of suspicion by determining which of the one or more regions of suspicion are the product of motion artifacts caused by the performance of motion correction; and
displaying the set of remaining regions of suspicion.

7. The method of claim 6, wherein the pre-contrast MR image and the sequence of post-contrast MR images comprise a dynamic contrast enhanced MRI.

8. The method of claim 6, wherein the sequence of post-contrast MR images are acquired at a regular interval of time after the administration of the contrast agent.

9. The method of claim 8, wherein the regular interval of time is one image per minute.

10. The method of claim 6, wherein the pre-contrast MR image includes T1 and T2 relaxation modalities.

11. The method of claim 6, wherein the sequence of post-contrast MR images include a T1 relaxation modality.

12. The method of claim 6, wherein the automatic identification of the regions of suspicion within the breast include identifying the regions of suspicion based on an absorption and washout profile observed from the sequence of post-contrast MR images.

13. The method of claim 6, wherein the pre-contrast MR image and the sequence of post-contrast MR images are corrected for magnetic field inhomogeneity prior to identifying the regions of suspicion.

14. The method of claim 6, wherein segmentation of the breast is performed on the pre-contrast MR image and the sequence of post-contrast MR images prior to identifying the regions of suspicion.

15. The method of claim 6, wherein one or more of the identified regions of interest are automatically characterized according to a BIRADS classification based on an absorption and washout profile for the respective identified region of suspicion observed from the sequence of post-contrast MR images.

16. The method of claim 6, wherein the step of removing one or more false positives from the one or more regions of suspicion include:
examining each identified region of suspicion;
calculating a measure of negative enhancement within a local neighborhood about each identified region of suspicion; and
removing each identified region of suspicion for which the calculated measure of negative enhancement is greater than a predetermined threshold.

17. A computer system comprising:
a processor; and
a program storage device readable by the computer system, embodying a program of instructions executable by the processor to perform method steps for automatically detecting breast lesions, the method comprising:
receiving a dynamic contrast enhanced MRI including a patient's breast on which motion correction has been performed;
automatically identifying one or more regions of suspicion within the breast;
removing one or more false positives from the one or more regions of suspicion to generate a set of remaining regions of suspicion by determining which of the one or more regions of suspicion are the product of motion artifacts caused by the performance of motion correction; and
displaying the set of remaining regions of suspicion.

18. The computer system of claim 17, wherein the dynamic contrast enhanced MRI includes a pre-contrast MR image and a sequence of post-contrast MR images acquired at a regular interval of time after administration of a magnetic contrast agent.

19. The computer system of claim 17, wherein the automatic identification of the regions of suspicion within the breast include identifying the regions of suspicion based on an absorption and washout profile observed from the dynamic contrast enhanced MRI.

20. The computer system of claim 17, wherein the step of removing one or more false positives from the one or more regions of suspicion include:
examining each identified region of suspicion;
calculating a measure of negative enhancement within a local neighborhood about each identified region of suspicion; and
removing each identified region of suspicion for which the calculated measure of negative enhancement is greater than a predetermined threshold.
